SEQUENCE LISTING (8 sequences)

SEQ ID NO: 1 - protein, 706 aa, Unknown
  Bacillus sp.
VFLKNLTVLLKTIPLALLLFILLSLPTAAQADVTNKVNYTRDVIYQIVTDRFSDGDPSNNPTGAIYSQDCSDLHKYCGGDWQGIIDKINDGYLTDLGITAIWISQPVENVYALHPSGYTSYHGYWARDYKRTNPFYGDFSDFDRLMDTAHSNGIKVIMDFTPNHSSPALETDPSYAENGAVYNDGVLIGNYSNDPNNLFHHNGGTDFSSYEDSIYRNLYDLADYDLNNTVMDQYLKESIKLWLDKGIDGIRVDAVKHMSEGWQTSLMSDIYAHEPVFTFGEWFLGSGEVDPQNHHFANESGMSLLDFQFGQTIRDVLMDGSSNWYDFNEMIASTEEDYDEVIDQVTFIDNHDMSRFSFEQSSNRHTDIALAVLLTSRGVPTIYYGTEQYLTGGNDPENRKPMSDFDRTTNSYQIISTLASLRQSNPALGYGNTSERWINSDVYIYERAFGDSVVLTAVNSGDTSYTINNLNTSLPQGQYTDELQQLLDGNEITVNSNGAVDSFQLSANGVSVWQITEEHASPLIGHVGPMMGKHGNTVTITGEGFGDNEGSVLFDSDFSDVLSWSDTKIEVSVPDVTAGHYDISVVNAGDSQSPTYDKFEVLTGDQVSIRFAVNNATTSLGTNLYMVGNVNELGNWDPDQAIGPMFNQVMYQYPTWYYDISVPAEENLEYKFIKKDSSGNVVWESGNNHTYTTPATGTDTVLVDWQ

SEQ ID NO: 2 - protein, 705 aa, Unknown
  Bacillus sp.
MLNKLSLKMKAIAFFGIVFVVFLALANDVYAANQLNKVNYAKDTIYQIVTDRFLDGDPSNNPDGALYSETDLHKYMGGDWKGITEKIEDHYFTDLGITALWISQPVENVYAVHPEGYTSYHGYWARDYKKTNPFYGNFNDFDELISTAHSHGIKIIMDFTPNHSSPALKTDSDYVENGAIYDNGSLIGNYSNDLDIFHHNGGTDFSSYEDGIYRNLYDLADYDLQNQTIDQYLKESIELWLDKGIDGIRVDAVKHMSQGWQETLTNHIYSYQPVFTFGEWFLGENEIDPRNHYFANESGMSLLDFQFGQQIRGVLMSQEDDWTDFHTMIEDTSNSYNEVIDQVTFIDNHDMSRFHKEDGAKTNTDIALAVLLTSRGVPTIYYGTEHYLTGESDPENRKPMPSFDRATTAYQIISKLAHLRQSNPALGYGTTTERWLNEDVYIFERKFGDNVVVTAVNSGEQSYTINNLQTSLLEGTHPDVLEGLMGGDALQIDGKGQASTFELKANEVAVWEVTAESNTPLIGHVGPMVGQAGNEITISGEGFGEGQGTVLFGSDQASIVSWGDSEIVVNVPDRPGNHYNIEVVTNDNKESNPYSDFEILTNKLIPVRFIVEEAVTDYGTSVYLVGNTQELGNWDTDKAIGPFFNQIIAQYPTWYYDISVPADSTLEYKFIKKDALGNVVWESGTNRSYETPTEGTDTLTSTWRN

SEQ ID NO: 3 - protein, 683 aa, Unknown
  Thermoanaerobacter sp.
APDTSVSNVVNYSTDVIYQIVTDRFLDGNPSNNPTGDLYDPTHTSLKKYFGGDWQGIINKINDGYLTGMGITAIWISQPVENIYAVLPDSTFGGSTSYHGYWARDFKKTNPFFGSFTDFQNLIATAHAHNIKVIIDFAPNHTSPASETDPTYGENGRLYDNGVLLGGYTNDTNGYFHHYGGTNFSSYEDGIYRNLFDLADLDQQNSTIDSYLKAAIKLWLDMGIDGIRMDAVKHMAFGWQKNFMDSILSYRPVFTFGEWYLGTNEVDPNNTYFANESGMSLLDFRFAQKVRQVFRDNTDTMYGLDSMIQSTAADYNFINDMVTFIDNHDMDRFYTGGSTRPVEQALAFTLTSRGVPAIYYGTEQYMTGNGDPYNRAMMTSFDTTTTAYNVIKKLAPLRKSNPAIAYGTQKQRWINNDVYIYERQFGNNVALVAINRNLSTSYYITGLYTALPAGTYSDMLGGLLNGSSITVSSNGSVTPFTLAPGEVAVWQYVSTTNPPLIGHVGPTMTKAGQTITIDGRGFGTTAGQVLFGTTPATIVSWEDTEVKVKVPALTPGKYNITLKTASGVTSNSYNNINVLTGNQVCVRFVVNNATTVWGENVYLTGNVAELGNWDTSKAIGPMFNQVVYQYPTWYYDVSVPAGTTIEFKFIKKNGSTVTWEGGYNHVYTTPTSGTATVIVDWQP

SEQ ID NO: 4 - protein, 713 aa, Unknown
  Bacillus sp.
MKRFMKLTAVWTLWLSLTLGLLSPVHAAPDTSVSNKQNFSTDVIYQIFTDRFSDGNPANNPTGAAFDGSCTNLRLYCGGDWQGIINKINDGYLTGMGITAIWISQPVENIYSVINYSGVNNTAYHGYWARDFKKTNPAYGTMQDFKNLIDTAHAHNIKVIIDFAPNHTSPASSDDPSFAENGRLYDNGNLLGGYTNDTQNLFHHYGGTDFSTIENGIYKNLYDLADLNHNNSSVDVYLKDAIKMWLDLGVDGIRVDAVKHMPFGWQKSFMATINNYKPVFTFGEWFLGVNEISPEYHQFANESGMSLLDFRFAQKARQVFRDNTDNMYGLKAMLEGSEVDYAQVNDQVTFIDNHDMERFHTSNGDRRKLEQALAFTLTSRGVPAIYYGSEQYMSGGNDPDNRARLPSFSTTTTAYQVIQKLAPLRKSNPAIAYGSTHERWINNDVIIYERKFGNNVAVVAINRNMNTPASITGLVTSLRRASYNDVLGGILNGNTLTVGAGGAASNFTLAPGGTAVWQYTTDATTPIIGNVGPMMAKPGVTITIDGRGFGSGKGTVYFGTTAVTGADIVAWEDTQIQVKIPAVPGGIYDIRVANAAGAASNIYDNFEVLTGDQVTVRFVINNATTALGQNVFLTGNVSELGNWDPNNAIGPMYNQVVYQYPTWYYDVSVPAGQTIEFKFLKKQGSTVTWEGGANRTFTTPTSGTATVNVNWQP

SEQ ID NO: 5 - protein, 719 aa, Bacillus stearothermophilus
  mat_peptide (34)..(719)
MKKKTLSLFVGLMLLIGLLFSGSLPYNPNAAEASSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 6 - protein, 534 aa, Aspergillus niger
  mat_peptide (25)..(534)
MSFRSLLALSGLVCTGLANVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKTSTTTRSGMSL

SEQ ID NO: 7 - protein, 478 aa, Aspergillus oryzae
ATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDITIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 8 - protein, 713 aa, Bacillus agaradhaerans
  mat_peptide (35)..(713)
MRKKTLKRLLTLVVGLVILSGLSILDFSITSASAQQATDRSNSVNYSTDVIYQIVTDRFYDGDESNNPSGELYSEDCKNLRKYCGGDWQGIIDKIDDGYLTNMGVTALWISPPVENIFETIDDEFGTTSYHGYWARDYKKTNPFFGSTEDFERLIETAHSHDIKIVIDLAPNHTSPADFDNPDYAENGVLYDDGNYLGSYSDDSDLFLYNGGTDFSNYEDEIYRNLFDLASFNHINSELNNYLEDAVKKWLDLGIDGIRIDAVAHMPPGWKKAYMDTIYDHRAVFTFGEWFTGPSGNEDYTKFANNSGMSVLDFRFAQTTRNVIGNNNGTMYDIEKMLTDTENDYDRPQDQVTFLDNHDMSRFTNGGESTRTTDIGLALMLTSRGVPTIYYGTEQYMKGDGDPGSRGMMASFDENTDAYKLIQKLAPLRKSNPAYGYGTTTERWINDDVLIYERHFGENYALIAINRSLNTSYNIQGLQTEMPSNSYDDVLDGLLDGQSIVVDNKGGVNEFQMSPGEVSVWEFEAENVDKPSIGQVGPIIGEAGRTVTISGEGFGSSQGTVHFGSTSAEILSWNDTIITLTVPNNEAGYHDITVVTEDEQVSNAYEFEVLTADQVTVRFIIDNAETKLGENVFLVGNVHELGNWDPEQSVGRFFNQIVYQYPTWYYDVNVPANTDLEFKFIKIDQDNNVIWQSGANQTYSSPESGTGIIRVDW

(19) United States
(12) Patent Application Publication
(10) Pub. No.: US 2009/0142817 A1
(43) Pub. Date: Jun. 4, 2009

(54) PROCESS FOR HYDROLYSIS OF STARCH

(75) Inventors: Barrie Edmund Norman, Birkerod (DK); Anders Vikso-Nielsen, Slangerup (DK); Hans Sejr Olsen, Holte (DK); Sven Pedersen, Gentofte (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/268,043

(22) Filed: Nov. 10, 2008

Related U.S. Application Data
(62) Division of application No. 10/504,543, filed on Aug. 13, 2004, now abandoned.
(60) Provisional application No. 60/358,507, filed on Feb. 19, 2002.

(30) Foreign Application Priority Data
Feb. 14, 2002 (DK) PA 2002 00227
Sep. 2, 2002 (DK) PA 2002 01291

(51) Int. Cl. C12P 7/06

(52) U.S. Cl. 435/161

(57) ABSTRACT

The present invention relates to a process for enzymatic hydrolysis of granular starch into a soluble starch hydrolyzate at a temperature below the initial gelatinization temperature of said granular starch.

### CROSS-REFERENCE TO RELATED APPLICATIONS

This application is a divisional of U.S. application Ser. No. 10/504,543 filed on Aug. 13, 2004, which is a 35 U.S.C. 371 national application of PCT/DK03/00084 filed Feb. 10, 2003, which claims priority or the benefit under 35 U.S.C. 119 of Danish application nos. PA 2002 00227 and PA 2002 01291 filed Feb. 14, 2002 and Sep. 2, 2002, respectively, and U.S. provisional application No. 60/358,507 filed Feb. 19, 2002, the contents of which are fully incorporated herein by reference.

### FIELD OF THE INVENTION

The present invention relates to a one step process for hydrolysis of granular starch into a soluble starch hydrolyzate at a temperature below the initial gelatinization temperature of said granular starch.

### BACKGROUND OF THE INVENTION

A large number of processes have been described for converting starch to starch hydrolyzates, such as maltose, glucose or specialty syrups, either for use as sweeteners or as precursors for other saccharides such as fructose. Glucose may also be fermented to ethanol or other fermentation products.

Starch is a high molecular-weight polymer consisting of chains of glucose units. It usually consists of about 80% amylopectin and 20% amylose. Amylopectin is a branched polysaccharide in which linear chains of alpha-1,4 D-glucose residues are joined by alpha-1,6 glucosidic linkages.

Amylose is a linear polysaccharide built up of D-glucopyranose units linked together by alpha-1,4 glucosidic linkages. In the case of converting starch into a soluble starch hydrolyzate, the starch is depolymerized. The conventional depolymerization process consists of a gelatinization step and two consecutive process steps, namely a liquefaction process and a saccharification process.

Granular starch consists of microscopic granules, which are insoluble in water at room temperature. When an aqueous starch slurry is heated, the granules swell and eventually burst, dispersing the starch molecules into the solution. During this “gelatinization” process there is a dramatic increase in viscosity. As the solids level is 30-40% in a typical industrial process, the starch has to be thinned or “liquefied” so that it can be handled. This reduction in viscosity is today mostly obtained by enzymatic degradation. During the liquefaction step, the long-chained starch is degraded into smaller branched and linear units (maltodextrins) by an alpha-amylase. The liquefaction process is typically carried out at about 105-110° C. for about 5 to 10 minutes followed by about 1-2 hours at about 95° C. The temperature is then lowered to 60° C., a glucoamylase or a beta-amylase and optionally a debranching enzyme, such as an isoamylase or a pullulanase are added, and the saccharification process proceeds for about 24 to 72 hours.

It will be apparent from the above discussion that the conventional starch conversion process is very energy consuming due to the different requirements in terms of temperature during the various steps. It is thus desirable to be able to select the enzymes used in the process so that the overall process can be performed without having to gelatinize the starch. Such processes are the subject for U.S. Pat. Nos. 4,591,560, 4,727,026 and 4,009,074 and EP 171218.

The present invention relates to a one-step process for converting granular starch into soluble starch hydrolyzate at a temperature below initial gelatinization temperature of the starch.

### SUMMARY OF THE INVENTION

In a first aspect the invention provides a one step process for producing a soluble starch hydrolyzate, the process comprising subjecting a aqueous granular starch slurry at a temperature below the initial gelatinization temperature of said granular starch to the simultaneous action of the following enzyme activities, a first enzyme which is a member of the Glycoside Hydrolase Family 13, has alpha-1.4-glucosidic hydrolysis activity and comprises a Carbohydrate-Binding Module of Family 20, and a second enzyme which is a fungal alpha-amylase (EC 3.2.1.1), a beta-amylase (E.C. 3.2.1.2), or an glucoamylase (E.C. 3.2.1.3).

In a second aspect the invention provides a process for production of high fructose starch-based syrup (HFSS), the process comprising producing a soluble starch hydrolyzate by the process of the first aspect of the invention, and further comprising a step for conversion of the soluble starch hydrolyzate into a of high fructose starch-based syrup (HFSS).

In a third aspect the invention provides a process for production of fuel or potable ethanol; comprising producing a soluble starch hydrolyzate by the process of the first aspect of the invention, and further comprising a step for fermentation of the soluble starch hydrolyzate into ethanol, wherein the fermentation step is carried out simultaneously or separately/sequential to the hydrolysis of the granular starch.

### DETAILED DESCRIPTION OF THE INVENTION

### Definitions

The term “granular starch” is understood as raw uncooked starch, i.e. starch that has not been subjected to a gelatinization. Starch is formed in plants as tiny granules insoluble in water. These granules are preserved in starches at temperatures below the initial gelatinization temperature. When put in cold water, the grains may absorb a small amount of the liquid. Up to 50° C. to 70° C. the swelling is reversible, the degree of reversibility being dependent upon the particular starch. With higher temperatures an irreversible swelling called gelatinization begins.

The term “initial gelatinization temperature” is understood as the lowest temperature at which gelatinization of the starch commences. Starch begins to gelatinize between 60° C. and 70° C., the exact temperature dependent on the specific starch. The initial gelatinization temperature depends on the source of the starch to be processed. The initial gelatinization temperature for wheat starch is approximately 52° C., for potato starch approximately 56° C., and for corn starch approximately 62° C. However, the quality of the starch initial may vary according to the particular variety of the plant species as well as with the growth conditions and therefore initial gelatinization temperature should be determined for each individual starch lot.

The term “soluble starch hydrolyzate” is understood as the soluble products of the processes of the invention and may comprise mono- di-, and oligosaccharides, such as glucose, maltose, maltodextrins, cyclodextrins and any mixture of these. Preferably at least 90%, 91%, 92%, 93%, 94%, 95%, 96%, 97% or 98% of the dry solids of the granular starch is converted into a soluble starch hydrolyzate.

The term “Speciality Syrups”, is an in the art recognised term and is characterised according to DE and carbohydrate spectrum (See the article “New Speciality Glucose Syrups”, p. 50+, in the textbook “Molecular Structure and Function of Food Carbohydrate”, Edited by G. G. Birch and L. F. Green, Applied Science Publishers LTD., London). Typically Speciality Syrups have a DE in the range from 35 to 45.

The “Glycoside Hydrolase Family 13” is in the context of this invention defined as the group of hydrolases comprising a catalytic domain having a (beta/alpha)₈ or TIM barrel structure and acting on starch and related substrates through an alpha-retaining reacting mechanism (Koshland, 1953, Biol. Rev. Camp. Philos. Soc. 28: 416-436).

The enzymes having “alpha-1.4-glucosidic hydrolysis activity” is in the context of this invention defined as comprising the group of enzymes which catalyze the hydrolysis and/or synthesis of alpha-1,4-glucosidic bonds as defined by Takata (Takata et al, 1992, J. Biol. Chem. 267: 18447-18452) and by Koshland (Koshland, 1953, Biol. Rev. Camp. Philos. Soc. 28: 416-436).

The “Carbohydrate-Binding Module of Family 20” or a CBM-20 module is in the context of this invention defined as a sequence of approximately 100 amino acids having at least 45% homology to the Carbohydrate-Binding Module (CBM) of the polypeptide having an amino acid sequence of SEQ ID NO: 3. The CBM comprises the last 102 amino acids of the polypeptide, i.e. the subsequence from amino acid 582 to amino acid 683.

Enzymes which (a) are members of the Glycoside Hydrolase Family 13; (b) have alpha-1.4-glucosidic hydrolysis activity; and (c) comprise a Carbohydrate-Binding Module of Family 20, and are specifically contemplated for this invention comprise the enzymes classified as EC 2.4.1.19, the cyclodextrin glucanotransferases, and EC 3.2.1.133, the maltogenic alpha-amylases, and selected members of 3.2.1.1 the alpha-amylases, and 3.2.1.60, the maltotetraose-forming amylases.

The “hydrolysis activity” of CGTases and maltogenic alpha-amylases is determined by measuring the increase in reducing power during incubation with starch according to Wind et al., 1995, Appl. Environ. Microbiol. 61:1257-1265. Reducing sugar concentrations is measured with the dinitrosalisylic acid method according to Bernfield (Bernfield, 1955, Amylases alpha and beta. Methods Enzymol. 1:149-158), with a few modifications. Diluted enzyme is incubated for an appropriate period of time with 1% (wt/v) soluble starch (Paselli SA2 starch from Avebe, The Netherlands or alternatively soluble starch from Merck) in a 10 mM sodium citrate (pH 5.9) buffer at 60° C. One unit of hydrolysis activity is defined as the amount of enzyme producing 1 micro mol of maltose per minute under standard conditions.

The polypeptide “homology” referred to in this disclosure is understood as the degree of identity between two sequences indicating a derivation of the first sequence from the second. The homology may suitably be determined by means of computer programs known in the art such as GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., 1970, Journal of Molecular Biology, 48: 443-453. The following settings for polypeptide sequence comparison are used: GAP creation penalty of 3.0 and GAP extension penalty of 0.1.

### Cyclodextrin Glucanotransferases (CGTases)

A particular enzyme to be used as a first enzyme in the processes of the invention may be a cyclomaltodextrin glucanotransferase (E.C. 2.4.1.19). Cyclomaltodextrin glucanotransferase, also designated cyclodextrin glucanotransferase or cyclodextrin glycosyltransferase, in the following termed CGTase, catalyses the conversion of starch and similar substrates into cyclomaltodextrins via an intramolecular transglycosylation reaction, thereby forming cyclomaltodextrins of various sizes. Most CGTases have both transglycosylation activity and starch-degrading activity. Contemplated CGTases are preferably of microbial origin, and most preferably of bacterial origin. Specifically contemplated CGTases include the CGTases having 50%, 55%, 60%, 65%, 70%, 75%, 80%, 85% or even 90% homology to the sequence shown as amino acids 1 to 679 of SEQ ID NO: 8, the CGTases having 50%, 55%, 60%, 65%, 70%, 75%, 80%, 85% or even 90% homology to the amino acid sequence of SEQ ID NO: 3, and the CGTases described in U.S. Pat. Nos. 5,278,059 and 5,545,587. Preferably the CGTase to be applied as a first enzyme of the process has a hydrolysis activity of at least 3.5, preferably at least 4, 4.5, 5, 6, 7, 8, 9, 10, 11, 12, 13, 14, 15, 16, 17, 18, 19, 20, 21, 22, or most preferably at least 23 micro mol per min/mg. CGTases may be added in amounts of 0.01-100.0 NU/g DS, preferably from 0.2-50.0 NU/g DS, preferably 10.0-20.0 NU/g DS.

### Maltogenic Alpha-Amylase

Another particular enzyme to be used as a first enzyme in the processes of the invention is a maltogenic alpha-amylase (E.C. 3.2.1.133). Maltogenic alpha-amylases (glucan 1,4-alpha-maltohydrolase) are able to hydrolyse amylose and amylopectin to maltose in the alpha-configuration. Furthermore, a maltogenic alpha-amylase is able to hydrolyse maltotriose as well as cyclodextrins. Specifically contemplated maltogenic alpha-amylases may be derived from Bacillus sp., preferably from Bacillus stearothermophilus, most preferably from Bacillus stearothermophilus C599 such as the one described in EP120.693. This particular maltogenic alpha-amylase has the amino acid sequence of amino acids 1-686 of SEQ ID NO: 5. A preferred maltogenic alpha-amylase has an amino acid sequence having at least 70% identity to amino acids 1-686 of SEQ ID NO: 5, preferably at least 80%, at least 85%, at least 90%, at least 92%, at least 95%, at least 96%, at least 97%, at least 98%, or particularly at least 99%. Most preferred variants of the maltogenic alpha-amylase comprise the variants disclosed in WO 99/43794.

The maltogenic alpha-amylase having the amino acid sequence of amino acids 1-686 of SEQ ID NO: 5 has a hydrolysis activity of 714. Preferably the maltogenic alpha-amylase to be applied as a first enzyme of the process has a hydrolysis activity of at least 3.5, preferably at least 4, 5, 6, 7, 8, 9, 10, 11, 12, 13, 14, 15, 16, 17, 18, 19, 20, 21, 22, 23, 100, 200, 300, 400, 500, 600, or most preferably at least 700 micro mol per min/mg.

Maltogenic alpha-amylases may be added in amounts of 0.01-40.0 MANU/g DS, preferably from 0.02-10 MANU/g DS, preferably 0.05-5.0 MANU/g DS.

### Fungal Alpha-Amylase

A particular enzyme to be used as a second enzyme in the processes of the invention is a fungal alpha-amylase (EC 3.2.1.1), such as a fungamyl-like alpha-amylase. In the present disclosure, the term “fungamyl-like alpha-amylase” indicates an alpha-amylase which exhibits a high homology, i.e. more than 50%, 55%, 60%, 65%, 70%, 75%, 80%, 85% or even 90% homology to the amino acid sequence of SEQ ID NO: 7. Fungal alpha-amylases may be added in an amount of 0.001-1.0 AFAU/g DS, preferably from 0.002-0.5 AFAU/g DS, preferably 0.02-0.1 AFAU/g DS.

### Beta-Amylase

Another particular enzyme to be used as a second enzyme in the processes of the invention may be a beta-amylase (E.C 3.2.1.2). Beta-amylase is the name traditionally given to exo-acting maltogenic amylases, which catalyze the hydrolysis of 1,4-alpha-glucosidic linkages in amylose, amylopectin and related glucose polymers.

Beta-amylases have been isolated from various plants and microorganisms (W. M. Fogarty and C. T. Kelly, 1979, Progress in Industrial Microbiology, 15: 112-115). These beta-amylases are characterized by having optimum temperatures in the range from 40° C. to 65° C. and optimum pH in the range from 4.5 to 7.0. Contemplated beta-amylases include the beta-amylase from barley Spezyme® BBA 1500, Spezyme® DBA and Optimalt™ ME, Optimalt™ BBA from Genencor Int. as well as Novozym™ WBA from Novozymes A/S.

### Glucoamylase

A further particular enzyme to be used as a second enzyme in the processes of the invention may also be a glucoamylase (E.C. 3.2.1.3) derived from a microorganism or a plant. Preferred is glucoamylases of fungal or bacterial origin selected from the group consisting of Aspergillus glucoamylases, in particular A. niger G1 or G2 glucoamylase (Boel et al., 1984, EMBO J. 3 (5): 1097-1102), or variants thereof, such as disclosed in WO 92/00381 and WO 00/04136; the A. awamori glucoamylase (WO 84/02921), A. oryzae (Agric. Biol. Chem., 1991, 55(4): 941-949), or variants or fragments thereof.

Other contemplated Aspergillus glucoamylase variants include variants to enhance the thermal stability: G137A and G139A (Chen et al., 1996, Prot. Engng. 9: 499-505); D257E and D293E/Q (Chen et al., 1995, Prot. Engng. 8: 575-582); N182 (Chen et al., 1994, Biochem. J. 301: 275-281); disulphide bonds, A246C (Fierobe et al., 1996, Biochemistry, 35: 8698-8704; and introduction of Pro residues in position A435 and S436 (Li et al., 1997, Protein Engng. 10: 1199-1204. Furthermore Clark Ford presented a paper on Oct. 17, 1997, ENZYME ENGINEERING 14, Beijing/China Oct. 12-17, 97, Abstract book p. 0-61. The abstract suggests mutations in positions G137A, N20C/A27C, and S30P in an Aspergillus awamori glucoamylase to improve the thermal stability. Other contemplated glucoamylases include Talaromyces glucoamylases, in particular derived from Talaromyces emersonii (WO 99/28448), Talaromyces leycettanus (U.S. Pat. No. Re.32,153), Talaromyces duponti, Talaromyces thermophilus (U.S. Pat. No. 4,587,215). Bacterial glucoamylases contemplated include glucoamylases from the genus Clostridium, in particular C. thermoamylolyticum (EP 135138), and C. thermohydrosulfuricum (WO 86/01831). Preferred glucoamylases include the glucoamylases derived from Aspergillus oryzae, such as a glucoamylase having 50%, 55%, 60%, 65%, 70%, 75%, 80%, 85% or even 90% homology to the amino acid sequence of SEQ ID NO: 6. Also contemplated are the commercial products AMG 200L; AMG 300 L; SANT™ SUPER and AMG™ E (from Novozymes); OPTIDEX™ 300 (from Genencor Int.); AMIGASE™ and AMIGASE™ PLUS (from DSM); G-ZYME™ G900 (from Enzyme Bio-Systems); G-ZYME™ G990 ZR (A. niger glucoamylase and low protease content).

Glucoamylases may be added in an amount of 0.02-2.0 AGU/g DS, preferably 0.1-1.0 AGU/g DS, such as 0.2 AGU/g DS.

### Additional Enzymes

The processes of the invention may also be carried out in the presence of a third enzyme. A particular third enzyme may be a Bacillus alpha-amylase (often referred to as “Termamyl-like alpha-amylases”). Well-known Termamyl-like alpha-amylases include alpha-amylase derived from a strain of B. licheniformis (commercially available as Termamyl), B. amyloliquefaciens, and B. stearothermophilus alpha-amylase. Other Termamyl-like alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., 1988, Biochemical and Biophysical Research Communications, 151: 2531. In the context of the present invention a Termamyl-like alpha-amylase is an alpha-amylase as defined in WO 99/19467 on page 3, line 18 to page 6, line 27. Contemplated variants and hybrids are described in WO 96/23874, WO 97/41213, and WO 99/19467. Specifically contemplated is a recombinant B. stearothermophilus alpha-amylase variant with the mutations: 1181*+G182*+N193F. Bacillus alpha-amylases may be added in effective amounts well known to the person skilled in the art.

Another particular third enzyme of the process may be a debranching enzyme, such as an isoamylase (E.C. 3.2.1.68) or a pullulanases (E.C. 3.2.1.41). Isoamylase hydrolyses alpha-1,6-D-glucosidic branch linkages in amylopectin and beta-limit dextrins and can be distinguished from pullulanases by the inability of isoamylase to attack pullulan, and by the limited action on alpha-limit dextrins. Debranching enzyme may be added in effective amounts well known to the person skilled in the art.

### EMBODIMENTS OF THE INVENTION

The starch slurry to be subjected to the processes of the invention may have 20-55% dry solids granular starch, preferably 25-40% dry solids granular starch, more preferably 30-35% dry solids granular starch.

After being subjected to the process of the first aspect of the invention at least 85%, 86%, 87%, 88%, 89%, 90%, 91%, 92%, 93%, 94%, 95%, 96%, 97%, 98%, or preferably 99% of the dry solids of the granular starch is converted into a soluble starch hydrolyzate.

According to the invention the processes of the first and second aspect is conducted at a temperature below the initial gelatinization temperature. Preferably the temperature at which the processes are conducted is at least 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or preferably at least 60° C.

The pH at which the process of the first aspect of the invention is conducted may in be in the range of 3.0 to 7.0, preferably from 3.5 to 6.0, or more preferably from 4.0-5.0.

The exact composition of the products of the process of the first aspect of the invention, the soluble starch hydrolyzate, depends on the combination of enzymes applied as well as the type of granular starch processed. Preferably the soluble hydrolyzate is maltose with a purity of at least 85%, 90%, 95.0%, 95.5%, 96.0%, 96.5%, 97.0%, 97.5%, 98.0%, 98.5, 99.0% or 99.5%. Even more preferably the soluble starch hydrolyzate is glucose, and most preferably the starch hydrolyzate has a DX (glucose percent of total solubilised dry solids) of at least 94.5%, 95.0%, 95.5%, 96.0%, 96.5%, 97.0%, 97.5%, 98.0%, 98.5, 99.0% or 99.5%. Equally contemplated, however, is the process wherein the product of the process of the invention, the soluble starch hydrolyzate, is a speciality syrup, such as a speciality syrup containing a mixture of glucose, maltose, DP3 and DPn for use in the manufacture of ice creams, cakes, candies, canned fruit.

The granular starch to be processed in the processes of the invention may in particular be obtained from tubers, roots, stems, legumes, cereals or whole grain. More specifically the granular starch may be obtained from corns, cobs, wheat, barley, rye, milo, sago, cassaya, tapioca, sorghum, rice, peas, bean, banana or potatoes. Specially contemplated are both waxy and non-waxy types of corn and barley. The granular starch to be processed may be a highly refined starch quality, preferably more than 90%, 95%, 97% or 99.5% pure or it may be a more crude starch containing material comprising milled whole grain including non-starch fractions such as germ residues and fibres. The raw material, such as whole grain, is milled in order to open up the structure and allowing for further processing. Two milling processes are preferred according to the invention: wet and dry milling. In dry milling the whole kernel is milled and used. Wet milling gives a good separation of germ and meal (starch granules and protein) and is with a few exceptions applied at locations where the starch hydrolyzate is used in production of syrups. Both dry and wet milling are well known in the art of starch processing and are equally contemplated for the processes of the invention. The process of the first aspect of the invention may be conducted in an ultrafiltration system where the retentate is held under recirculation in presence of enzymes, raw starch and water and where the permeate is the soluble starch hydrolyzate. Equally contemplated is the process conducted in a continuous membrane reactor with ultrafiltration membranes and where the retentate is held under recirculation in presence of enzymes, raw starch and water and where the permeate is the soluble starch hydrolyzate. Also contemplated is the process conducted in a continuous membrane reactor with microfiltration membranes and where the retentate is held under recirculation in presence of enzymes, raw starch and water and where the permeate is the soluble starch hydrolyzate.

In the process of the second aspect of the invention the soluble starch hydrolyzate of the process of the first aspect of the invention is subjected to conversion into high fructose starch-based syrup (HFSS), such as high fructose corn syrup (HFCS). This conversion is preferably achieved using a glucose isomerase, and more preferably by an immobilized glucose isomerase supported on a solid support. Contemplated isomerases comprises the commercial products Sweetzyme™ IT from Novozymes A/S, G-zyme™ IMGI and G-zyme™ G993, Ketomax™ and G-zyme™ G993 from Rhodia, G-zyme™ G993 liquid and GenSweet™ IGI from Genencor Int.

In the process of the third aspect of the invention the soluble starch hydrolyzate of the process of the first aspect of the invention is used for production of fuel or potable ethanol. In the process of the third aspect the fermentation may be carried out simultaneously or separately/sequential to the hydrolysis of the granular starch slurry. When the fermentation is performed simultaneous to the hydrolysis the temperature is preferably between 30° C. and 35° C., and more preferably between 31° C. and 34° C. The process of the third aspect of the invention may be conducted in an ultrafiltration system where the retentate is held under recirculation in presence of enzymes, raw starch, yeast, yeast nutrients and water and where the permeate is an ethanol containing liquid. Equally contemplated is the process conducted in a continuous membrane reactor with ultrafiltration membranes and where the retentate is held under recirculation in presence of enzymes, raw starch, yeast, yeast nutrients and water and where the permeate is an ethanol containing liquid.

### Materials and Methods

### Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue colour is formed, but during the break-down of the starch the blue colour gets weaker and gradually turns into a reddish-brown, which is compared to a coloured glass standard.

One Kilo Novo alpha amylase Unit (KNU) is defined as the amount of enzyme which, under standard conditions (i.e. at 37° C.+/−0.05; 0.0003 M Ca²⁺; and pH 5.6) dextrinizes 5.26 g starch dry substance Merck Amylum solubile.

A folder AF 9/6 describing this analytical method in more detail is available upon request to Novozymes A/S, Denmark, which folder is hereby included by reference.

### CGTase Activity (KNU)

The CGTase alpha-amylase activity is determined by a method employing Phadebas® tablets as substrate. Phadebas tablets (Phadebas® Amylase Test, supplied by Pharmacia Diagnostic) contain a cross-linked insoluble blue-colored starch polymer, which has been mixed with bovine serum albumin and a buffer substance.

For every single measurement one tablet is suspended in a tube containing 5 ml 50 mM Britton-Robinson buffer (50 mM acetic acid, 50 mM phosphoric acid, 50 mM boric acid, 0.1 mM CaCl2, pH adjusted to the value of interest with NaOH). The test is performed in a water bath at the temperature of interest. The alpha-amylase to be tested is diluted in x ml of 50 mM Britton-Robinson buffer. 1 ml of this alpha-amylase solution is added to the 5 ml 50 mM Britton-Robinson buffer. The starch is hydrolyzed by the alpha-amylase giving soluble blue fragments. The absorbance of the resulting blue solution, measured spectrophotometrically at 620 nm, is a function of the alpha-amylase activity.

It is important that the measured 620 nm absorbance after 10 or 15 minutes of incubation (testing time) is in the range of 0.2 to 2.0 absorbance units at 620 nm. In this absorbance range there is linearity between activity and absorbance (Lambert-Beer law). The dilution of the enzyme must therefore be adjusted to fit this criterion. Under a specified set of conditions (temperature, pH, reaction time, buffer conditions) 1 mg of a given alpha-amylase will hydrolyze a certain amount of substrate and a blue colour will be produced. The colour intensity is measured at 620 nm. The measured absorbance is directly proportional to the specific activity (activity/mg of pure alpha-amylase protein) of the alpha-amylase in question under the given set of conditions.

A folder EAL-SM-0351 describing this analytical-method in more detail is available upon request to Novozymes A/S, Denmark, which folder is hereby included by reference.

### Maltogenic Alpha-Amylase Activity (MANU)

One Maltogenic Amylase Novo Unit (MANU) is defined as the amount of enzyme which under standard will cleave one micro mol maltotriose per minute. The standard conditions are 10 mg/ml maltotriose, 37° C., pH 5.0, and 30 minutes reaction time. The formed glucose is converted by glucose dehydrogenase (GlucDH, Merck) to gluconolactone under formation of NADH, which is determined spectophotometrically at 340 nm. A folder (EAL-SM-0203.01) describing this analytical method in more detail is available on request from Novozymes A/S, Denmark, which folder is hereby included by reference.

### Glucoamylase Activity (AGU)

The Novo Glucoamylase Unit (AGU) is defined as the amount of enzyme, which hydrolyzes 1 micromole maltose per minute at 37° C. and pH 4.3.

The activity is determined as AGU/ml by a method modified after (AEL-SM-0131, available on request from Novozymes) using the Glucose GOD-Perid kit from Boehringer Mannheim, 124036. Standard: AMG-standard, batch 7-1195, 195 AGU/ml. 375 microL substrate (1% maltose in 50 mM Sodium acetate, pH 4.3) is incubated 5 minutes at 37° C. 25 microL enzyme diluted in sodium acetate is added. The reaction is stopped after 10 minutes by adding 100 microL 0.25 M NaOH. 20 microL is transferred to a 96 well microtitre plate and 200 microL GOD-Perid solution (124036, Boehringer Mannheim) is added. After 30 minutes at room temperature, the absorbance is measured at 650 nm and the activity calculated in AGU/ml from the AMG-standard. A folder (AEL-SM-0131) describing this analytical method in more detail is available on request from Novozymes A/S, Denmark, which folder is hereby included by reference.

### Fungal Alpha-Amylase Activity (FAU)

The alpha-amylase activity is measured in FAU (Fungal Alpha-Amylase Units). One (1) FAU is the amount of enzyme which under standard conditions (i.e. at 37° C. and pH 4.7) breaks down 5260 mg solid starch (Amylum solubile, Merck) per hour. A folder AF 9.1/3, describing this FAU assay in more details, is available upon request from Novozymes A/S, Denmark, which folder is hereby included by reference.

### Acid Alpha-Amylase Activity (AFAU)

Acid alpha-amylase activity is measured in AFAU (Acid Fungal Alpha-amylase Units), which are determined relative to an enzyme standard.

The standard used is AMG 300 L (from Novozymes A/S, glucoamylase wildtype Aspergillus niger G1, also disclosed in Boel et al., 1984, EMBO J. 3 (5): 1097-1102 and in WO 92/00381). The neutral alpha-amylase in this AMG falls after storage at room temperature for 3 weeks from approx. 1 FAU/mL to below 0.05 FAU/mL.

The acid alpha-amylase activity in this AMG standard is determined in accordance with the following description. In this method 1 AFAU is defined as the amount of enzyme, which degrades 5.26 mg starch dry solids per hour under standard conditions.

Iodine forms a blue complex with starch but not with its degradation products. The intensity of colour is therefore directly proportional to the concentration of starch. Amylase activity is determined using reverse colorimetry as a reduction in the concentration of starch under specified analytic conditions.

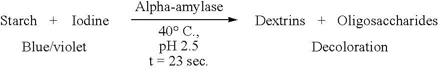

Standard conditions/reaction conditions: (per minute)

| | |
|---|---|
| Substrate: | starch, approx. 0.17 g/L |
| Buffer: | Citate, approx. 0.03 M |
| Iodine (I2): | 0.03 g/L |
| CaCl2: | 1.85 mM |
| pH: | 2.50-0.05 |
| Incubation temperature: | 40° C. |
| Reaction time: | 23 seconds |
| Wavelength: | lambda = 590 nm |
| Enzyme concentration: | 0.025 AFAU/mL |
| Enzyme working range: | 0.01-0.04 AFAU/mL |

If further details are preferred these can be found in EBSM-0259.02/01 available on request from Novozymes A/S, and incorporated by reference.

### Beta-Amylase Activity (DP°)

The activity of SPEZYME® BBA 1500 is expressed in Degree of Diastatic Power (DP°). It is the amount of enzyme contained in 0.1 ml of a 5% solution of the sample enzyme preparation that will produce sufficient reducing sugars to reduce 5 ml of Fehling's solution when the sample is incubated with 100 ml of substrate for 1 hour at 20° C.

### Pullulanase Activity (New Pullulanase Unit Novo (NPUN)

Pullulanase activity may be determined relative to a pullulan substrate. Pullulan is a linear D-glucose polymer consisting essentially of maltotriosyl units joined by 1,6-alpha-links. Endo-pullulanases hydrolyze the 1,6-alpha-links at random, releasing maltotriose, 6³-alpha-maltotriosyl-maltotriose, 6³-alpha-(6³-alpha-maltotriosyl-maltotriosyl)-maltotriose.

One new Pullulanase Unit Novo (NPUN) is a unit of endo-pullulanase activity and is measured relative to a Novozymes A/S Promozyme D standard. Standard conditions are 30 minutes reaction time at 40° C. and pH 4.5; and with 0.7% pullulan as substrate. The amount of red substrate degradation product is measured spectrophotometrically at 510 nm and is proportional to the endo-pullulanase activity in the sample. A folder (EB-SM.0420.02/01) describing this analytical method in more detail is available upon request to Novozymes A/S, Denmark, which folder is hereby included by reference.

Under the standard conditions one NPUN is approximately equal to the amount of enzyme which liberates reducing carbohydrate with a reducing power equivalent to 2.86 micromole glucose per minute.

### Determination of CGTase Hydrolysis Activity

The CGTase hydrolysis activity was determined by measuring the increase in reducing power during incubation with Paselli SA2 starch (from Avebe, The Netherlands) as described by Wind et al., 1995, Appl. Environ. Microbiol. 61: 1257-1265.

### Determination of Sugar Profile and Solubilised Dry Solids

The sugar composition of the starch hydrolyzates was determined by HPLC and glucose yield was subsequently calculated as DX. °BRIX, solubilized (soluble) dry solids of the starch hydrolyzates were determined by refractive index measurement.

### Materials

The following enzyme activities were used. A maltogenic alpha-amylase with the amino acid sequence of amino acids 1-686 of SEQ ID NO: 5. A glucoamylase derived from Aspergillus oryzae having the amino acid sequence of SEQ ID NO: 6. An acid fungal alpha-amylase derived from Aspergillus niger. A Bacillus alpha-amylase which is a recombinant B. stearothermophilus variant with the mutations: 1181*+G182*+N193F. A fungal alpha-amylase derived from Aspergillus oryzae. A CGTase N with the sequence shown herein as SEQ ID NO 1. A CGTase O with the sequence of SEQ ID NO: 2. A CGTase T with the amino acid sequence of SEQ ID NO: 3. A CGTase A having the sequence of SEQ ID NO: 4.

Common corn starch (C×PHARM 03406) was obtained from Cerestar.

### Example 1

This example illustrates the conversion of granular starch into glucose using CGTase T and a glucoamylase and an acid fungal amylase. A slurry with 33% dry solids (DS) granular starch was prepared by adding 247.5 g of common corn starch under stirring to 502.5 ml of water. The pH was adjusted with HCl to 4.5. The granular starch slurry was distributed to 100 ml blue cap flasks with 75 g in each flask. The flasks were incubated with magnetic stirring in a 60° C. water bath. At zero hours the enzyme activities given in table 1 were dosed to the flasks. Samples were withdrawn after 24, 48, 72, and 96 hours.

TABLE 1
The enzyme activity levels used were:

| | | Acid fungal |
|---|---|---|
| CGTase T | Glucoamylase | alpha-amylase |
| KNU/kg DS | AGU/kg DS | AFAU/kg DS |
| 12.5 | 200 | 50 |
| 25.0 | 200 | 50 |
| 100.0 | 200 | 50 |

Total dry solids starch was determined using the following method. The starch was completely hydrolyzed by adding an excess amount of alpha-amylase (300 KNU/Kg dry solids) and subsequently placing the sample in an oil bath at 95° C. for 45 minutes. After filtration through a 0.22 microM filter the dry solids was measured by refractive index measurement.

Soluble dry solids in the starch hydrolyzate were determined on samples after filtering through a 0.22 microM filter. Soluble dry solids were determined by refractive index measurement and the sugar profile was determined by HPLC. The amount of glucose was calculated as DX. The results are shown in tables 2 and 3.

TABLE 2
Soluble dry solids as percentage of total dry
substance at the three CGTase activity levels.

| KNU/kg DS | 24 hours | 48 hours | 72 hours | 96 hours |
|---|---|---|---|---|
| 12.5 | 68 | 82 | 89 | 94 |
| 25.0 | 76 | 89 | 93 | 97 |
| 100.0 | 83 | 96 | 98 | 99 |

TABLE 3
The DX of the soluble hydrolyzate at
the three CGTase activity levels.

| KNU/kg DS | 24 hours | 48 hours | 72 hours | 96 hours |
|---|---|---|---|---|
| 12.5 | 92.6 | 94.5 | 95.1 | 95.3 |
| 25.0 | 92.4 | 94.8 | 95.4 | 95.5 |
| 100.0 | 92.7 | 94.9 | 95.4 | 95.4 |

### Example 2

This example illustrates the conversion of granular starch into glucose using CGTase T, a glucoamylase, an acid fungal alpha-amylase and a Bacillus alpha-amylase.

Flasks with 33% DS granular starch were prepared and incubated as described in example 1. At zero hours the enzymes activities given in table 4 were dosed to the flask.

TABLE 4
The enzyme activity levels used were:

| | | Acid fungal | Bacillus |
|---|---|---|---|
| CGTase T | Glucoamylase | alpha-amylase | alpha-amylase |
| KNU/kg DS | AGU/kg DS | AFAU/kg DS | KNU/kg DS |
| 5.0 | 200 | 50 | 300 |

Samples were withdrawn after 24, 48, 72, and 96 hours and analyzed as described in example 1. The results are shown in tables 4 and 5.

TABLE 5
Soluble dry solids as percentage of total dry substance.

| 24 hours | 48 hours | 72 hours | 96 hours |
|---|---|---|---|
| 82.8 | 93.0 | 96.3 | 98.7 |

TABLE 6
The DX of the soluble hydrolyzate.

| 24 hours | 48 hours | 72 hours | 96 hours |
|---|---|---|---|
| 92.8 | 94.9 | 95.5 | 95.8 |

### Example 3

This example illustrates the conversion of granular starch into glucose using a maltogenic alpha-amylase, a glucoamylase and an acid fungal alpha-amylase.

Flasks with 33% DS granular starch were prepared and incubated as described in example 1. At zero hours the enzyme activities given in table 6 were dosed to the flasks.

TABLE 6
The enzyme activity levels used were:

| | Maltogenic | | Acid fungal |
|---|---|---|---|
| | alpha-amylase | Glucoamylase | alpha-amylase |
| | MANU/kg DS | AGU/kg DS | AFAU/kg DS |
| Flask 1 | 5000 | 200 | 50 |
| Flask 2 | 20000 | 200 | 50 |

Samples were withdrawn after 24, 48, 72, and 96 hours and analyzed as described in example 1. The results are shown in table 7 and 8.

TABLE 7
Soluble dry solids as percentage of total dry substance
at the two maltogenic alpha-amylase activity levels.

| MANU/kg DS | 24 hours | 48 hours | 72 hours | 96 hours |
|---|---|---|---|---|
| 5000 | 63.1 | 75 | 79.3 | 85.3 |
| 20000 | 67.0 | 77.9 | 82.7 | 88.1 |

TABLE 8
The DX of the soluble hydrolyzate at the two
maltogenic alpha-amylase activity levels.

| MANU/kg DS | 24 hours | 48 hours | 72 hours | 96 hours |
|---|---|---|---|---|
| 5000 | 95.2 | 95.4 | 95.3 | 95.5 |
| 20000 | 93.8 | 94.9 | 94.9 | 94.8 |

### Example 4

This example illustrates the only partial conversion of granular starch into glucose using a glucoamylase and an acid fungal alpha-amylase.

Flasks with 33% DS granular starch were prepared and incubated as described in example 1. At zero hours the enzyme activities given in table 9 were dosed to the flasks. Samples were withdrawn after 24, 48, 72, and 96 hours. The samples were analyzed as described in examples 1. The results are shown in tables 10 and 11.

TABLE 9
The enzyme activity level used were:

| Glucoamylase | Acid fungal alpha-amylase |
|---|---|
| AGU/kg DS | AFAU/kg DS |
| 200 | 50 |

TABLE 10
Soluble dry solids as percentage of total dry substance.

| 24 hours | 48 hours | 72 hours | 96 hours |
|---|---|---|---|
| 28.5 | 36.3 | 41.6 | 45.7 |

TABLE 11
DX of the soluble hydrolyzate.

| 24 hours | 48 hours | 72 hours | 96 hours |
|---|---|---|---|
| 27.7 | 34.9 | 39.2 | 42.2 |

### Example 5

This example illustrates the correlation between the hydrolysis activity of four different CGTases (CGTase A, CGTase N, CGTase O and CGTase T) versus the yield during conversion of granular starch into glucose syrup using a CGTase and a glucoamylase measured as soluble dry solids and development in DX.

Flasks with 33% DS granular starch were prepared and incubated as described in example 1. At zero hour the CGTases were all dosed at 100 KNU/kg DS in combination with glucoamylase at 200 AGU/kg DS. Samples were withdrawn at 48 hours and analyzed as described in examples 1. Results are presented in table 12.

TABLE 12
Hydrolysis activity (micro mol per min/mg protein),
and soluble dry solids (DS) and DX after 48 hors

| CGTase | Hydrolysis act. | Soluble DS | DX |
|---|---|---|---|
| CGTase N | 0.27 | 37.4 | 35.1 |
| CGTase A | 0.38 | 49.9 | 46.7 |
| CGTase O | 1.62 | 60.9 | 57.1 |
| CGTase T | 4.59 | 97.9 | 91.2 |

### Example 6

This example illustrates the process conducted in an ultrafiltration system where the retentate was held under recirculation in presence of enzymes, raw starch and water and where the permeate is the soluble starch hydrolyzate. A slurry comprising 100 kg granular corn starch suspended in 233 L tap city water and CGTase T (12.5 KNU/kg starch), Bacillus alpha-amylase (300 KNU/kg starch) and glucoamylase (200 AGU/kg starch) was processed in a batch ultrafiltration system (type PCI) with a tubular membrane module (type PU 120). The slurry was stirred at 100 rpm, pH was adjusted to 4.5 using 170 mL of 30% HCl, and the reaction temperature was set at 57° C.

Samples of permeate and retentate were analyzed for dry solids content and for sugar composition.

The correction factor for non soluble material is: q=(100-S %)/(100-°BRIX). The centrifugation index for sugar is: ciS %=°BRIX/S % (no correction). The theoretical yield of sugar (glucose) S_{yield}=ciS %*q*100/111*100%. A correction has thus been done for 100 kg starch dry matter giving ca. 111 kg glucose dry matter as a result of the hydrolysis reaction.

A trial was made in a simple batch system using the same enzyme system as for the membrane trial. As the comparison in table 15 a and b shows the membrane system reached the maximal solubilization of starch earlier.

TABLE 13
Dry solids content and sugar composition
of retentate and permeate

| | | Reactor | % | % | % | % | % |
|---|---|---|---|---|---|---|---|
| Sample | Hours | volume, L | DS | DP1 | DP2 | DP3 | DP4 |
| Reactor | 3 | 207 | 16.1 | 75.3 | 10.3 | 2.6 | 11.5 |
| Reactor | 28 | 123 | 28.3 | 95.0 | 2.7 | 0.8 | 1.5 |
| Reactor | 53 | 123 | 31.4 | 95.2 | 3.4 | 0.5 | 0.9 |
| Permeate | 3 | 207 | 12.1 | 71.2 | 17.4 | 2.9 | 8.5 |
| Permeate | 28 | 123 | 21.8 | 94.9 | 2.9 | 0.8 | 1.3 |

TABLE 14
Dry solids distribution in retentate at 3, 28, 53 and 77

| | 3 hours | 28 hours | 53 hours | 77 hours |
|---|---|---|---|---|
| Soluble DS | 16 | 28 | 31 | 39 |
| Total DS | 38 | 37 | 42 | 45 |

TABLE 15 a
Theoretical yield of glucose versus time for the membrane system

| | | | | | Theoretical |
|---|---|---|---|---|---|
| | % total DS | | q = (100 − | | yield sis = |
| | in the | | S %)/(100 − | cis % = ° | cis*q*100/ |
| Hours | reactor | ° BRIX | ° Brix) | Brix/S % | 111 % |
| 0 | 27.0 | 2.2 | 0.75 | 0.08 | 5 |
| 24 | 35.9 | 27.3 | 0.88 | 0.76 | 73 |
| 48 | 41.2 | 30.0 | 0.84 | 0.73 | 89 |
| 72 | 41.2 | 33.1 | 0.88 | 0.80 | 98 |
| 94 | 41.2 | 34.8 | 0.90 | 0.85 | 103 |

TABLE 15 b
Theoretical yield of glucose versus
time for a batch reactor system.

| | | | | | Theoretical |
|---|---|---|---|---|---|
| | % total DS | | q = (100 − | | yield sis = |
| | in the | | S %)/(100 − | cis % = ° | cis*q*100/ |
| Hours | reactor | ° BRIX | ° Brix) | Brix/S % | 111 % |
| 0 | 29.7 | 2.0 | 0.72 | 0.07 | 4. |
| 24 | 29.7 | 25.6 | 0.95 | 0.86 | 74 |
| 48 | 29.7 | 28.8 | 0.99 | 0.97 | 86 |
| 72 | 29.7 | 29.8 | 1.00 | 1.00 | 91 |
| 94 | 29.7 | 29.8 | 1.00 | 1.00 | 91 |

The conclusion was that when substrate saturation was maintained during the saccharification in a membrane system the degree of solubilization was improved compared to a simple batch reactor system for cold saccharification of raw starch.

### Example 7

This example illustrates a simultaneous cold liquefaction and saccharification process of the invention carried out in a continuous working microfiltration membrane reactor using a ceramic module.

A 200 L feed mixer tank was connected by a reactor feed pump to a 200 L reactor tank with temperature control. Using a pump with a capacity of 0-20 l/h the mixture from the reactor was recycled through an APV ceramic microfiltration module for separation of glucose. Pore size was 0.2 micro m and the membrane area was 0.2 m².

The reactor worked for about 200 hours using a dosage of 100 KNU/kg DS CGTase T and 300 AGU/kg DS of glucoamylase. With an average holding time in the reactor of 35-45 hours the system operated at steady state for the full period producing a DP1=93% glucose syrup at a yield of close to 100%.

The reactor tank was loaded with 60 kg of corn starch type Cerestar C×PHARM 03406 suspended in 140 L of tap city water of 58° C. under stirring. Using the steam heated mantel the temperature was adjusted to 60° C. Using 30% HCl the pH was lowered from 6.1 to 4.5. The pH was re-checked (pH=4.5) after 15 minutes.

At zero hours, immediately before adding the enzymes, CGTase T (100 KNU/kg starch) and glucoamylase (300 AGU/kg starch), samples were taken for determination of % sludge volume after centrifugation at 3000 rpm for 3 min in a table centrifuge. Furthermore the °BRIX of the supernatant was measured using a refractometer. The course of the reaction was followed regularly by measurements of sludge volumes and °BRIX of the supernatants as described above.

The feed mixer tank was loaded with 186 L of cold tap city water and 80 kg corn starch type Cerestar C×PHARM 03406. The feed mixer was kept stirred gentle and pH was adjusted to 4.5 using 30% HCl. The temperature was kept at 7-8° C. using cooling water and the enzymes CGTase T (100 KNU/kg starch) and glucoamylase (300 AGU/kg starch) was added. The low temperature secured that no reaction took place.

The upstart of the reactor was continued until the °Brix-value after 30 hours had stabilized around 27. Then the microfiltration was initiated using a pressure drop of 0.15 Bar and maximal retentate flow to secure this pressure. The filtrate was recycled to the reactor tank the first 5.7 hours. Hereafter the filtrate was collected in a separate tank, and the volume was measured as a function of time. At this point of time the reactor feed pump was started and adjusted to a flow rate equivalent to the filtrate flux (L/min). By doing so the volume in the reactor tank was kept constant.

The feed of starch slurry was continued while samples were taken as described above. Furthermore samples of the filtrates were taken. Any decrease in the filtrate flux was compensated for by increasing the retentate flow whereby the filter cake on the membrane was disrupted. Thereby the pressure drop was increased too. Samples were taken as a function of time of the filtrate for HPLC and °BRIX as well as the volume collected was measured. Simultaneously samples were taken from the reactor for measuring of total DS, sludge, °Brix and HPLC for sugar composition.

The trial lasted 220 hours. At that point of time the pressure drop was increased to about 0.4 Bar.

Determination of filtrate flux (based on single determinations) and average filtrate flux values (integrated) as a function of the process time showed that the enzyme system consisting of a CGTase and a glucoamylase alone maintained and secured a stable flux over a long processing time. This underlines the industrial potential advantages of this stable system.

The results and a mass balance are presented in tables 16-18.

TABLE 16
Analyses of collected filtrates.

| | Hours | | | | | Average |
|---|---|---|---|---|---|---|
| | from | Collected | % DS | Density, | Mass of | flux, |
| Date and time | Start | filtrate, L | w/w | kg/L | DS, Kg | mL/min |
| 13/03/02 16:05 | 30* | — | — | — | — | — |
| 14/03/02 16:50 | 55 | 142 | 25.8 | 1.12 | 41.1 | 95.6 |
| 16/03/02 16:00 | 102 | 187 | 25.6 | 1.12 | 53.7 | 66.1 |
| 18/03/02 13:02 | 147 | 200 | 28.7 | 1.14 | 65.2 | 74.0 |
| 19/03/02 16:45 | 174 | 100 | 29.6 | 1.14 | 33.8 | 60.1 |
| Total collected | | 629.0 | 27.3 | 1.13 | 193.7 | — |

*Start of continuous feeding to the reactor

TABLE 17
Composition of the syrup produced

| % DP1 | % DP2 | % DP3 | % DP4 |
|---|---|---|---|
| 93 | 5 | 1 | 2 |

TABLE 18
Mass balance for the trial of example 7

| | | | |
|---|---|---|---|
| Mass, | % | Mass of | % Yield |
| kg | DS | DS, kg | of DS* |

Upstart of reactor

| | | | | |
|---|---|---|---|---|
| Starch | 60 | 90 | 54 | 25 |
| Water | 140 | 0 | 0 | |
| Reactor start | 200 | 27.0 | 54 | 25 |

Continuous production

| | | | | |
|---|---|---|---|---|
| Starch consumption | | | | |
| (t = 28.75 h to t = 174.5 h) | 235.48 | 90 | 212 | 100 |
| Water consumption | 548.12 | 0 | 0 | |
| (t = 28.75 h to t = 174.5 h) | | | | |
| Substrate consumption | 783.6 | 27.0 | 212 | 100 |
| Syrup production | 629.0 | 27.3 | 172 | 81 |

Reactor at end

| | | | | |
|---|---|---|---|---|
| Total content | 200 | 35 | 70 | 33 |
| Unconverted starch | 18 | 50 | 9 | 4 |
| Mud, L | 18 | 50 | 9 | 4 |
| Glucose syrup | 164 | 30 | 49 | 23 |

*basis substrate consumption at continuous production.

Compared to a batch trial carried out in a simple tank with stirring a significant reduction of the reaction time was obtained using the setup for hydrolysis of granular starch described above. As no viscosity problems were encountered with 30% DS it is considered feasible to increase the DS to 40%, or even as high as 45% and still maintain a smooth operation.

### Example 8

This example compares a process of the invention and a conventional process for production of fuel ethanol or potable alcohol from raw starch in the form of dry milled corn, Yellow Dent No. 2.

A slurry of 30% DS of dry milled corn was prepared in tap water in 250 ml blue cap flasks and the raw corn starch exposed to simultaneous cold liquefaction and pre-saccharification by a process of the invention. The slurry was heated to 60° C. in a water bath under magnet stirring, pH adjusted to 4.5 using 30% HCl and CGTase T (75 KNU/kg DS) and glucoamylase (500 AGU/kg DS) added. After 48 hours the flask was cooled in the water bath to 32° C.

A slurry of 30% DS dry milled corn was pre-liquefied in a conventional continuous process consisting of a pre-liquefaction vessel, a jet-cooker, a flash, and a post liquefaction vessel. Bacillus alpha-amylase was added during the pre-liquefaction at 70-90° C. (10 KNU/kg DS) and again during the post liquefaction at ca. 85-90° C. (20 KNU/kg DS). The jet-cooking was carried out at 115-120° C. Pre-saccharification was performed under magnet stirring by heating the mash in blue cap flasks to 60° C. in a water bath. After pH adjustment to 4.5 using 30% HCl glucoamylase was added in a dosage equivalent to 500 AGU/kg DS. After 48 hours the flask was cooled in the water bath to 32° C.

Fermentations were made directly in the blue cap flasks fitted with yeast locks filled with soybean oil. Bakers yeast (Saccharomyces cerevisiae) was added in an amount equivalent to 10 millions/mL of viable yeast cells and yeast nutrition in the form of 0.25% urea was added to each flask. Each treatment was performed in 3 replicates.

The fermentation was monitored by the CO₂ loss as determined by weighing the flasks at regular intervals. L EtOH/100 kg grain dry matter (DS) was then calculated using the following formula:

$$L Et OH \text{/} 100 kg mash dry matter = \frac{Weight loss ( g ) \times 1.045}{0.79 ( g \text{/} mL ) \times 250 \times 30 \% dry matter} \times 100$$

The mash contained 30% w/w grain dry matter. 0.79 g/mL is the density of ethanol.

Tables 19 and 20 shows the obtained fermentation results for the replicates including the results of statistical calculation of the two types of pretreated raw materials (missing results estimated by interpolation).

TABLE 19
Fermentation result for the process of the invention using
CGTase T (75 KNU/kg DS) and glucoamylase (500 AGU/kg DS).

| Hour | L EtOH/100 kg grain | STDEV |
|---|---|---|
| 0 | — | — |
| 25.5 | 28.3 | 0.9 |
| 48 | 35.4 | 0.6 |
| 69 | 37.1 | 0.2 |
| 79 | *37.5 | — |
| 97 | 38.3 | 0.2 |

*Estimated value

TABLE 20
Fermentation result for a conventional process
using Bacillus alpha-amylase (10 + 20 KNU/kg
DS) and glucoamylase (500 AGU/kg DS)

| Hour | L EtOH/100 kg grain | STDEV |
|---|---|---|
| 0 | — | — |
| 25.5 | 22.5 | 1.3 |
| 48 | 33.9 | 0.7 |
| 69 | *37.2 | — |
| 79 | 38.8 | 0.4 |
| 97 | 40.5 | 0.5 |

*Estimated value

Using a simulated industrial fermentation time in the interval of approximately 48-70 hours an equivalent or higher alcohol yield was obtained from the mash produced by the process of the invention than could be obtained from a mash produced by the more energy consuming two step hot slurry pre-liquefying and jet-cooking process.

### Example 9

This example illustrates the conversion of granular wheat and common corn starch into glucose using a CGTase, a glucoamylase and an acid fungal alpha-amylase at 60° C.

Flasks with either 33% DS common corn or wheat granular starch were prepared and incubated as described in example 1. At zero hours the enzyme activities given in table 20 were dosed to the flasks. Samples were withdrawn after 24, 48, 72, and 96 hours and analyzed as described in example 1. The results are shown in table 21 and table 22.

TABLE 20
The enzyme activity levels used were:

| | | Acid fungal |
|---|---|---|
| CGTase | Glucoamylase | alpha-amylase |
| NU/g DS | AGU/g DS | AFAU/g DS |
| 100.0 | 0.2 | 0.05 |

TABLE 21
Soluble dry solids as percentage of total dry
substance using two different starch types.

| Starch | 24 hours | 48 hours | 72 hours | 96 hours |
|---|---|---|---|---|
| Common corn | 85.9 | 96.2 | 99.4 | 100.0 |
| Wheat | 95.7 | 98.9 | 99.6 | 100.0 |

TABLE 22
The DX of the soluble hydrolyzate using
the two different starch types.

| Starch | 24 hours | 48 hours | 72 hours | 96 hours |
|---|---|---|---|---|
| Common corn | 76.2 | 89.2 | 93.4 | 94.7 |
| Wheat | 86.2 | 92.4 | 93.6 | 94.4 |

1-34. (canceled)

35. A process for producing ethanol, comprising:
(a) subjecting an aqueous granular starch slurry at a temperature below the initial gelatinization temperature of said granular starch to the simultaneous action of an alpha-amylase (EC 3.2.1.1) which comprises a carbohydrate binding module family 20 and one or more enzymes selected from the group consisting of beta-amylase and glucoamylase to produce a soluble starch hydrolyzae; and
(b) fermenting the soluble starch hydrolyzate into ethanol.

36. The process of claim 35, wherein the starch slurry has 20-55% dry solids granular starch.

37. The process of claim 35, wherein at least 85% of the dry solids of the granular starch is converted into the soluble starch hydrolyzate.

38. The process of claim 35, wherein the carbohydrate binding module comprises amino acids 582 to 683 of SEQ ID NO: 3.

39. The process of claim 35, further comprising subjecting the aqueous granular starch slurry with a fungal alpha-amylase.

40. The process of claim 35, wherein the enzyme is a beta-amylase.

41. The process of claim 35, wherein the enzyme is a barley beta-amylase.

42. The process of claim 35, wherein the enzyme is a glucoamylase.

43. The process of claim 35, wherein the enzyme is a glucoamylase derived from Aspergillus oryzae.

44. The process of claim 35, further comprising subjecting the aqueous granular starch slurry with an alpha-amylase derived from a Bacillus sp.

45. The process of claim 35, further comprising subjecting the aqueous granular starch slurry with an isoamylase or a pullulanase.

46. The process of claim 35, wherein the temperature is at least 58° C.

47. The process of claim 35, wherein the pH is in the range of 3 to 7.

48. The process of claim 35, wherein the soluble starch hydrolyzate has a DX of at least 94.5%.

49. The process of claim 35, wherein the granular starch is obtained from tubers, roots, stems, or whole grain.

50. The process of claim 35, wherein the granular starch is obtained from cereals.

51. The process of claim 35, wherein the granular starch is obtained from corn, cobs, wheat, barley, rye, milo, sago, cassaya, tapioca, sorghum, rice or potatoes.

52. The process of claim 35, wherein the granular starch is obtained from dry milling of whole grain or from wet milling of whole grain.

53. The process of claim 35, wherein the fermentation is carried out simultaneously or separately/sequential to the hydrolysis of the granular starch.
